(19) United States
(12) Patent Application Publication
(10) Pub. No.: US 2020/0170983 A1
(43) Pub. Date: Jun. 4, 2020

(54) METHODS FOR THE TREATMENT OF BLADDER CANCER

(71) Applicant: Cipla Limited, Mumbai (IN)

(72) Inventors: Geena Malhotra, Mumbai (IN); Kalpana Joshi, Maharashtra (IN)

(21) Appl. No.: 16/166,305

(22) Filed: Oct. 22, 2018

Related U.S. Application Data
(63) Continuation of application No. 15/384,960, filed on Dec. 20, 2016, now abandoned.

(30) Foreign Application Priority Data
Dec. 23, 2015 (IN) 4836/MUM/2015

(51) Int. Cl. A61K 31/192; A61K 45/06; A61K 33/24

(52) U.S. Cl. CPC A61K 31/192; A61K 33/24; A61K 45/06

(57) ABSTRACT

Methods of treating bladder cancer using adapalene are disclosed herein. Adapalene can be administered as part of a comprehensive treatment program, which can also include chemotherapy, immunotherapy, radiation therapy and/or surgical treatment.

### CROSS REFERENCE TO RELATED APPLICATION

This application is a continuation of U.S. application Ser. No. 15/384,960, filed Dec. 20, 2016, which claims the benefit of Indian Application 4836/MUM/2015, filed Dec. 23, 2015, the contents of which are incorporated herein by reference.

### FIELD OF THE INVENTION

The present invention is directed to methods of treating bladder cancer using adapalene or a pharmaceutically acceptable salt thereof.

### BACKGROUND

Bladder cancer is a life-threatening and progressive disease, which usually begins in the lining of the epithelial lining (i.e., the urothelium) of the urinary bladder. Invasive bladder cancer may spread to lymph nodes, other organs in the pelvis (causing problems with kidney and bowel function), or other organs in the body, such as the liver and lungs. Standard treatments for bladder cancer are surgery, radiation therapy, chemotherapy, and biological therapy

Bladder cancer is diagnosed using cystoscopy and/or cytology, however, the latter is not very sensitive—a negative result cannot reliably exclude bladder cancer. Cigarette smoking and various environmental and occupational exposures are the major risk factors for bladder cancer. These substances concentrate in the urine, where the urothelial lining is exposed to their carcinogenic effects. Cigarette smoking is associated with up to 50% to 60% of bladder cancer diagnosed in men and 30% among women in the United States. Occupational exposure among white men accounts for 25% of bladder cancer diagnoses in men and 11% in women. Specific chemicals linked to bladder carcinogenesis include beta-naphthylamine, 4-aminobiphenyl, and benzidine. Evidence also suggests that chlorinated organic compounds formed as a by-product of drinking water chlorination may account for 10% to 15% of cases. Infection with Schistosoma haematobium is a well-documented risk factor and an important cause of bladder cancer in developing countries.

In the United States, more than 90% of cancers arising in the bladder are urothelial carcinoma (UC), also known as transitional cell carcinoma (TCC). Less common pathologies are squamous cell carcinoma (SCC), adenocarcinoma, and small cell carcinoma, comprising approximately <10% of bladder tumors, respectively. In Egypt, SCC comprises 70% of all bladder cancers.

Macroscopically, papillary growth is more frequent than solid tumors (approximately 80% versus 20%). Solid tumors are more likely than papillary tumors to be high grade and invasive into the muscularis propria layer.

There are newer non-invasive urine bound markers available as aids in the diagnosis of bladder cancer, including human complement factor H-related protein, high-molecular-weight carcinoembryonic antigen, and nuclear matrix protein 22 (NMP22). NMP22 is also available as a prescription home test. Other non-invasive urine based tests include the CertNDx Bladder Cancer Assay, which combines FGFR3 mutation detection with protein and DNA methylation markers to detect cancers across stage and grade, UroVysion, and Cxbladder. The diagnosis of bladder cancer can also be done with a Hexvix/Cysview guided fluorescence cystoscopy (blue light cystoscopy, Photodynamic diagnosis), as an adjunct to conventional white-light cystoscopy. This procedure improves the detection of bladder cancer and reduces the rate of early tumor recurrence, compared with white light cystoscopy alone. Cysview cystoscopy detects more cancer and reduces recurrence. Cysview is marketed in Europe under the brand name Hexvix.

Although the collective term “superficial cancer” is still commonly used, it is a category that comprises several classes of transitional cell cancers with different rates of recurrence, different rates of progression to muscle invasion, and quite different treatments. The tumors that arise in the epithelium and develop in an exophytic (papillary) pattern are known as Ta tumors. They are usually low grade (I or II), and although they tend to recur, they are considered to be relatively benign lesions that closely resemble the normal urothelium. Although they have more than the normal seven layers of urothelium, they show normal nuclear polarity in more than 95% of tumors and no (or slight) pleomorphism. When progression deeper into the submucosa or lamina propria occurs the tumor is described as T1 and carries a higher risk of progression and even of metastasis. Grade is an important predictor of recurrence and progression for all categories of superficial disease. Pathologic grades I to III (low, intermediate, or high) are based on the number of mitoses, presence of nuclear abnormalities, and cellular atypia. High-grade tumors show loss of polarization of the nuclei and moderate to prominent pleomorphism. Muscle-invasive disease, however, is usually high grade, and depth of invasion is the more important prognostic factor for outcome. Carcinoma in situ (Tcis or cis or Tis) is defined as noninvasive, high-grade, flat cancer confined to the epithelium, which can be localized or diffuse, and it may occur in association with either superficial or muscle-invasive TCC. In T2 lesions, muscle invasion is present and the probability of nodal and distant spread is increased. T2 disease is divided into superficial (T2a) or deep (T2b) invasion. SCCs are associated with chronic inflammation or infection with Schistosoma and tend to grow as large masses with a high degree of necrosis.

Stage 0 bladder cancer includes non-invasive papillary carcinoma (Ta) and flat non-invasive carcinoma (Tis). In either case, the cancer has not invaded the bladder wall beyond the inner layer. This early stage of bladder cancer is most often treated with transurethral resection (TUR). This may be followed either by observation (close follow-up without further treatment) or by intravesical therapy to try to keep the cancer from coming back.

Of the intravesical treatments, Bacille-Calmette Guerin (BCG) seems to be better at both keeping cancers from coming back and from getting worse. But it also tends to have more side effects. For this reason, doctors usually reserve BCG for cancers that are more likely to come back as invasive cancer or spread within the bladder.

Stage 0a: For low-grade non-invasive papillary (Ta) tumors, the options after TUR include observation, a single dose of intravesical chemotherapy (usually mitomycin) within a day of surgery, or weekly intravesical chemo, starting a few weeks after surgery. If the cancer comes back, the treatments can be repeated.

High-grade non-invasive papillary (Ta) tumors are more likely to come back after treatment, so intravesical BCG is often recommended after surgery. Another option is intravesical chemotherapy with mitomycin. As with BCG, this is usually started several weeks after surgery and is given every week for several weeks. A third option is close observation without intravesical treatment.

Stage 0is: For flat non-invasive (Tis) tumors, BCG is the treatment of choice after surgery. Patients with these tumors often get 6 weekly treatments of intravesical BCG, starting a few weeks after TUR. Some doctors recommend repeating BCG treatment every 3 to 6 months. BCG treatment reduces the recurrence rate by at least half.

Stage 0 bladder cancers rarely need to be treated with partial or radical cystectomy. Cystectomy is considered only when there are many superficial cancers or when a superficial cancer continues to grow (or seems to be spreading) despite treatment.

Following treatment for any stage 0 cancer, close follow-up is recommended, with cystoscopy about every 3 to 6 months for a least a couple of years to look for signs of the cancer coming back or for new bladder tumors.

The outlook for people with stage 0a (non-invasive papillary) bladder cancer is excellent. These cancers are nearly always cured with the right treatment. During long-term follow-up care, more superficial cancers are often found in the bladder or elsewhere in the urinary system. Although these new cancers do need to be treated, they rarely are deeply invasive or life threatening.

The long-term outlook for stage 0is (flat non-invasive) bladder cancer is not quite as good as for stage 0a cancers. These cancers have a higher risk of coming back, and may return as a more serious cancer, one that is growing into deeper layers of the bladder or has spread to other tissues.

Stage I bladder cancers have grown into the connective tissue layer of the bladder wall but have not reached the muscle layer.

Transurethral resection (TUR) is typically the initial treatment for these cancers. Over half of these patients later get a new bladder cancer. In many cases, the new cancer will invade the bladder muscle and be a higher stage. This is more likely to happen if the first cancer is high grade.

Even if the cancer is found to be low grade, a second TUR may be recommended several weeks later. If the doctor feels that all of the cancer has been removed, intravesical BCG or mitomycin is given. If the doctor was not able to remove all of the cancer, options include either intravesical BCG or cystectomy (removal of part or all of the bladder).

If the cancer is high grade, if many tumors are present, or if the tumor is very large when it is first found, radical cystectomy may be recommended. This is done to try to keep the cancer from coming back and spreading elsewhere. Another option for some high-grade tumors may be a repeat transurethral resection (TUR) followed by intravesical BCG.

For people who can't have a cystectomy, radiation therapy (often along with chemo) may be an option as the main treatment, although the chances for cure may not be as good.

These cancers have invaded the muscle layer of the bladder wall. Transurethral resection (TUR) is typically the first treatment for these cancers, but it is done to help determine the extent of the cancer rather than to try to cure it.

When the cancer has invaded the muscle, radical cystectomy is the standard treatment. Lymph nodes near the bladder are often removed as well. If cancer is in only one part of the bladder, some patients can be treated with a partial cystectomy instead. Only a small number of patients are good candidates for this.

Although at this stage cancer cells have not been detected outside the bladder, in some cases there may already be tiny deposits of cancer, called micrometastases, growing elsewhere in the body. These are too small to see on imaging tests but may eventually grow and become life threatening. This risk is greater with more deeply invasive cancers and higher-grade cancers. For this reason, chemotherapy is often given either before surgery (neoadjuvant chemo) or after surgery (adjuvant chemo) to lower the chance the cancer will come back in a distant site.

Many doctors prefer to give chemo before surgery because it has been shown to help patients live longer than surgery alone. When chemo is given first, surgery is delayed. This is not a problem if the chemotherapy causes the bladder cancer to shrink, but it might be harmful if the tumor continues to grow during chemotherapy.

Another option for some patients may be transurethral resection (TUR), followed by radiation and chemotherapy. Some people may prefer this because it lets them keep their bladder, but it's not clear if the outcomes are as good as they are after cystectomy, so not all doctors agree with this approach. If this treatment is used you will need frequent and careful follow-up exams. Some experts recommend a repeat cystoscopy and biopsy during treatment with chemo and radiation. If cancer is found in the biopsy sample, a cystectomy will likely be needed.

For patients who cannot have a major operation because of other serious medical conditions, TUR, radiation, or chemotherapy may be used as the only treatment. If the patient is well enough, chemotherapy may be given along with radiation therapy to help it work better.

Stage III cancers have reached the outside of the bladder and might have grown into nearby tissues or organs.

Transurethral resection (TUR) is typically done first to help determine the extent of the cancer. Radical cystectomy and removal of nearby lymph nodes is then the standard treatment. Partial cystectomy is seldom an option for stage III cancers.

Neoadjuvant chemotherapy is often given before surgery. It can shrink the tumor, which may make surgery easier. This can be especially useful for T4a tumors, which have grown outside the bladder. The chemotherapy may also kill any cancer cells that could already have spread to other areas of the body. This approach helps patients live longer than cystectomy alone. When chemotherapy is given first, surgery to remove the bladder is delayed. The delay is not a problem if the chemotherapy causes the bladder cancer to shrink, but it can be harmful if the tumor continues to grow during chemotherapy.

Some patients get chemotherapy after surgery (adjuvant treatment) to kill any areas of cancer cells left after surgery that are too small to see. Chemotherapy given after cystectomy may help patients stay cancer-free longer, but so far it's not clear if it helps them live longer.

Some patients with single, small T3a tumors can be treated with a transurethral resection (TUR) of the tumor followed by a combination of chemotherapy and radiation. If this isn't successful and cancer is found when cystoscopy is repeated, the patient might need cystectomy.

For patients who cannot have a major operation because of other serious medical conditions, TUR, radiation, or chemotherapy may be used as the only treatment. If the patient is well enough, chemotherapy may be given along with radiation therapy to help it work better.

Stage IV cancers have reached the abdominal or pelvic wall (T4b tumors) or have spread to nearby lymph nodes or distant parts of the body.

In most cases surgery (even radical cystectomy) cannot remove all of the cancer at this stage, so these cancers are very hard to get rid of completely. Treatment is usually aimed at slowing the cancer's growth and spread to help you live longer and feel better. If you and your doctor discuss surgery as treatment option, be sure you understand the goal of the operation—whether it is to try to cure the cancer, to help you live longer, or to help prevent or relieve symptoms from the cancer—before deciding on treatment.

For stage IV bladder cancers that have not spread to distant sites, chemotherapy (with or without radiation) is usually the first treatment. If the cancer shrinks in response to treatment, a cystectomy might be an option. Patients who can't tolerate chemotherapy (because of other health problems) are often treated with radiation therapy.

For stage IV bladder cancers that have spread to distant areas, chemotherapy is usually the first treatment, sometimes along with radical cystectomy or radiation therapy. Patients who can't tolerate chemotherapy (because of other health problems) are often treated with radiation therapy. Urinary diversion without cystectomy is sometimes done to prevent or relieve a blockage of urine that could otherwise cause severe kidney damage.

In more severe cases, a partial or radical cystectomy may be performed. However, because of concerns regarding recurrence, patients often receive chemotherapy or immunotherapy in addition to surgery. Chemotherapies which have been employed include methotrexate, vinblastine, doxorubicin, and cisplatin (MVAC), gemcitabine and cisplatin (GC). Administration of these drugs is often accompanied by severe negative side effects.

Immunotherapies include intravesicular delivery of Bacillus Calmette-Guérin (BCG). BCG is a vaccine against tuberculosis that is prepared from attenuated (weakened) live bovine tuberculosis bacillus, Mycobacterium bovis that has lost its virulence in humans. BCG immunotherapy is effective in up to 66% of the cases at this stage, and in randomized trials has been shown to be superior to standard chemotherapy. The mechanism by which BCG prevents recurrence is unknown, but the presence of bacteria in the bladder may trigger a localized immune reaction which clears residual cancer cells. However, bladder cancer recurring in patients subsequent to BCG treatment is more difficult to treat.

There remains a need for effective, non-surgical treatments of bladder cancer, including bladder cancer recurring post-BCG treatment. There remains a need for agents effective to treat bladder cancer with reduced side effect profiles relative to currently used medications.

### SUMMARY

Disclosed herein are methods of treating bladder cancer, including bladder cancer recurring post-BCG treatment. The methods include administering to a patient in need thereof adapalene in an amount effective to treat the bladder cancer. In some instances, adapalene can be administered as part of a combination therapy. Also disclosed herein are pharmaceutical compositions containing adapalene suitable for the treatment of bladder cancer. In some instances, the compositions include an additional anti-cancer agent.

The details of one or more embodiments are set forth in the descriptions below. Other features, objects, and advantages will be apparent from the description and from the claims.

### BRIEF DESCRIPTION OF THE FIGURES

FIG. 1 includes a depiction of a 2D assay of adapalene against human bladder cancer cells.

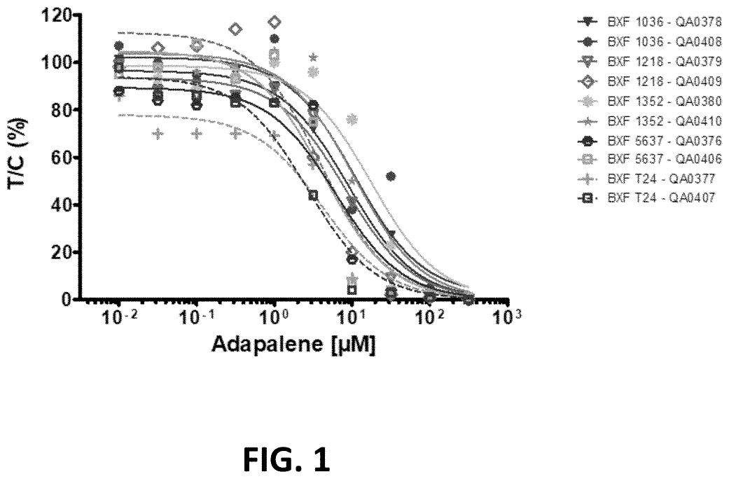

FIG. 2 includes a depiction of a 2D assay of cisplatin against human bladder cancer cells.

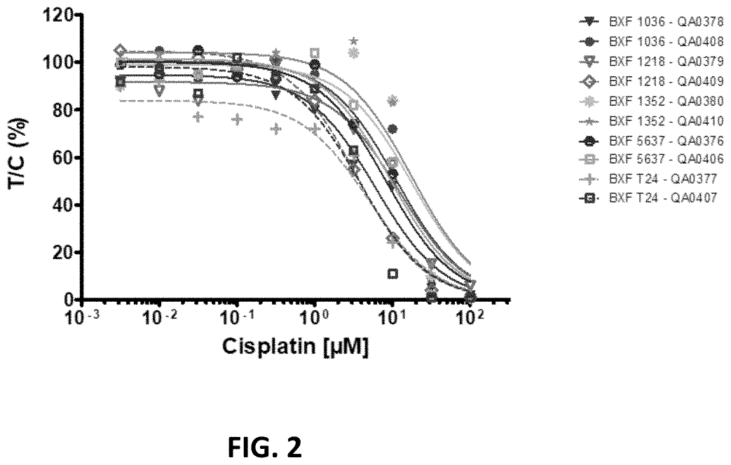

FIG. 3 includes a depiction of a 3D assay of adapalene against human bladder cancer cells.

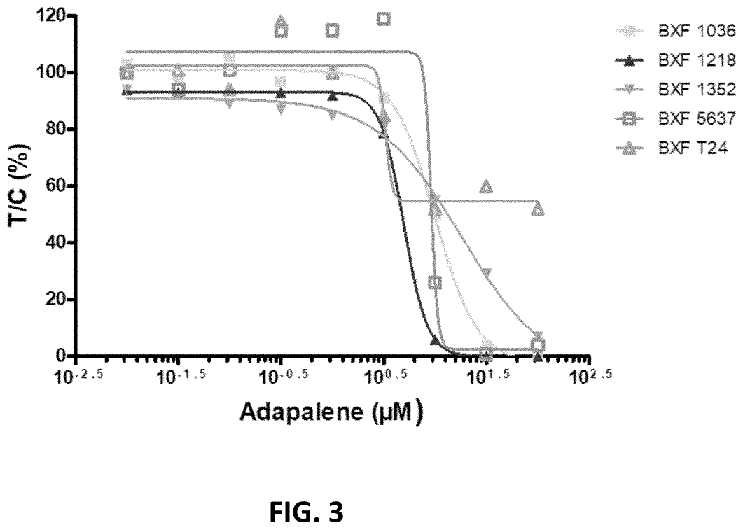

FIG. 4 includes a depiction of a 3D assay of cisplatin against human bladder cancer cells.

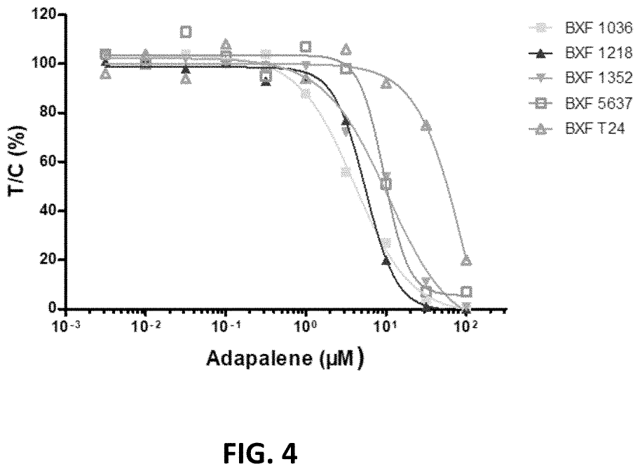

FIG. 5 includes a depiction of the anti-tumor efficacy of adapalene in combination with cisplatin in BXF 1036L; A) Modeled T/C, which is the mean of experimental T/C for each pair of conditions in the combination matrix. B) Bliss index, which is the difference of Bliss neutral and modeled T/C for each pair of conditions.

FIG. 6 includes a depiction of the anti-tumor efficacy of adapalene in combination with cisplatin in BXF 1218L. A) Modeled T/C, which is the mean of experimental T/C for each pair of conditions in the combination matrix. B) Bliss index, which is the difference of Bliss neutral and modeled T/C for each pair of conditions.

FIG. 7 includes a depiction of the anti-tumor efficacy of adapalene in combination with cisplatin in BXF T-24. A) Modeled T/C, which is the mean of experimental T/C for each pair of conditions in the combination matrix. B) Bliss index, which is the difference of Bliss neutral and modeled T/C for each pair of conditions

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Adapalene is a third-generation topical retinoid primarily used in the treatment of mild-moderate acne, and is also used off-label to treat keratosis pilaris as well as other skin conditions. Adapalene is a synthetic naphthoic acid derivative with retinoid activity. Adapalene may be represented by the following chemical formula:

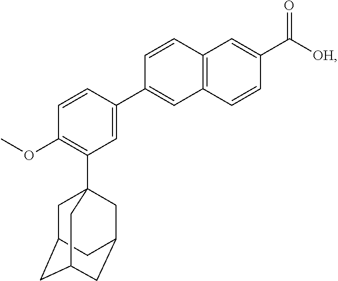 [SMILES: COc1ccc(-c2ccc3cc(C(=O)O)ccc3c2)cc1C12CC3CC(CC(C3)C1)C2]

Nuclear retinoid receptors are the proximate mediators of many of the effects of retinoids on gene expression. Two types of receptors have been identified: retinoic acid receptors (RARs) and retinoid X receptors (RXRs). The RARs bind to ATRA and 9-cis-retinoic acid (9cRA), a natural retinoic acid isomer, which binds to both RARs and RXRs. RARs can form heterodimers with RXRs and bind to retinoic acid response elements, specific DNA sequences that are characterized by direct repeats of (A/G)GGTCA separated by two or five nucleotides that act as ligand-dependent transcriptional regulators for retinoic acid-responsive genes

Retinoids, including ATRA, 4-HPR (or Fenretinide), (or AHPN) have been studied for different receptor binding preferences, and based on the study, the synthetic retinoids have exhibited stronger effects on growth inhibition and apoptosis induction in bladder cancer cells than the natural one.

The inventors of the present invention have found that adapalene plays a very strong role in inducing apoptosis in bladder cancer cells by exhibiting selective agonist activity on retinoic acid receptors RARβ and RARγ.

Disclosed herein are methods of treating bladder cancer in a patient in need thereof by administering an effective amount of adapalene. Unless stated to the contrary, the term adapalene refers both to adapalene free acid and pharmaceutically acceptable salts thereof.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, ptoluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Preferably, adapalene may be administered to the subject once daily, twice daily or thrice daily. A typical recommended daily dosage regimen can range from about 5 mg to 2,000 mg, from about 0.1 mg to 500 mg, from about 10 mg to 1,000 mg, from about 10 mg to 500 mg, from about 10 mg to 400 mg, from about 10 to 200 mg, from about 10 to 100 mg, from about 10 to 50 mg, from about 50 to 400 mg, from about 100 to 400 mg, or from about 200 to 400 mg. In other embodiments, the total daily dosage can be from about 5 mg to 5,000 mg, from about 10 mg to 4,000 mg, from about 100 mg to 4,000 mg, from about 500 mg to 4,000 mg, from about 500 to 2,000 mg, from about 1,000 to 2,000 mg, from about 1,000 to 3,000 mg, from about 1,500 to 2,500 mg, from about 500 to 1,500 mg, or from about 2,000 to 4,000 mg.

Preferably, the active agent may be provided in the form of a pharmaceutical composition such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution and sprinkles, transdermal patches, however, other dosage forms such as controlled release formulations, lyophilized formulations, modified release formulations, delayed release formulations, extended release formulations, pulsatile release formulations, dual release formulations and the like. Liquid and semisolid dosage forms (liquids, suspensions, solutions, dispersions, ointments, creams, emulsions, microemulsions, sprays, patches, spot-on), parenteral, topical, inhalation, buccal, nasal etc. may also be envisaged under the ambit of the invention. The inventors of the present invention have also found that the solubility properties of the active agent may be improved by nanosizing thus leading to better bioavailability and dose reduction of the drug.

In one embodiment, adapalene may be present in the form of nanoparticles which have an average particle size of less than 2,000 nm, less than 1,500 nm, less than 1,000 nm, less than 750 nm, less than 500 nm, or less than 250 nm.

Suitable excipients may be used for formulating the dosage form according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

Depending on the pathological stage, patient's age and other physiological parameters, and the extent of cancer progression, adapalene may require specific dosage amounts and specific frequency of administrations. Preferably, the active agent may be administered at least once, twice or thrice a day in an amount from 0.1 to 500 mg or 10 mg to 2,000 mg. In some embodiments, the active agent may be administered such that the total daily dose is in an amount from 10-1,000 mg, 50-1,000 mg, 50-750 mg, 50-500 mg, 100-500 mg, 250-2,000 mg, 500-2,000 mg, 500-1,000 mg, 250-1,000 mg, 250-500 mg, 1,000-2,000 mg, or 1,500-2,000.

Adapalene can be used to treat bladder cancers. In some embodiments, adapalene can reduce tumor size, inhibit tumor growth, alleviate symptoms, delay progression, prolong survival, including, but not limited to disease free survival, prevent or delay bladder cancer metastasis, reduce or eliminate preexisting bladder cancer metastasis, and/or prevent recurrence of bladder cancer.

As used herein, the term “delay” refers to methods that reduce the probability of disease development/extent in a given time frame, when compared to otherwise similar methods that do not include the use of adapalene. Probabilities can be established using clinical trials, but can also be determined using in vitro assays when correlations have been established. In some embodiments, adapalene can inhibit bladder cancer cell proliferation. For instance, at least about 10%, 20%, 30%, 40%, 60%, 70%, 80%, 90%, or 100% of cell proliferation is inhibited. In some embodiments, adapalene can inhibit bladder cancer metastasis. For instance, at least about 10%, 20%, 30%, 40%, 60%, 70%, 80%, 90%, or 100% of metastasis is inhibited.

It can be preferable to diagnose the patient with bladder cancer prior to commencing the therapeutic methods disclosed herein. When the functional consequences are determined using intact cells or animals, one can also measure a variety of effects such as, in the case of bladder cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian bladder cancer polypeptide is typically used, e.g., mouse, preferably human. Tumor cells release an increased amount of certain factors (hereinafter “tumor specific markers”) than their normal counterparts. For example, plasminogen activator (PA) is released from human glioma at a higher level than from normal brain cells. See, e.g., “Angiogenesis, tumor vascularization, and potential interference with tumor growth” pp. 178-184 in Mihich (ed. 1985) Biological Responses in Cancer Plenum. Similarly, tumor angiogenesis factor (TAF) is released at a higher level in tumor cells than their normal counterparts. See, e.g., Folkman (1992) Sem Cancer Biol. 3:89-96. Different urine tests are available to look for specific substances released by bladder cancer cells. One or more of these tests may be used along with urine cytology to help determine the bladder cancer. These include the tests for NMP22 (BladderChek) and BTA (BTA stat), the Immunocyt test, and the UroVysion test. In other instances, the patient can be diagnosed with bladder cancer using cystoscopy. In certain embodiments, a patient having detectable amount of one or more of the above markers, after receiving adapalene, with exhibit a 10%, 20%, 30%, 40%, 60%, 70%, 80%, 90%, or 100% reduction in that marker.

Bladder cancer can be characterized by overall stage, 0-IV. Stage 0 is refined by the letters a (designating non-invasive papillary carcinoma) and is (designating non-invasive flat carcinoma, which can be referred to as CIS). The stages can be further refined by one of three categories: T categories refer to the extent the tumor has grown into or beyond the wall of the bladder. T1 refers to cancer that has not grown into the muscle layers of the bladder. T2a indicates the cancer has grown into the inner half of the muscle layer, while T2b indicates the outer half of the muscle layer has been compromised. T3 indicates the tumor has grown into the fatty tissue surrounding the bladder (T3a refers to tumors that are only detectable by microscope, while T3b indicates the tumor can be seen or felt by a physician). T4a indicates the tumor has grown into the stroma of the prostate in men, and into either the uterus or vagina in women. T4b indicates the tumor has reached the pelvic or abdominal wall. N categories refer to the spread in the lymph nodes near the pelvis and along the common iliac artery—N0: There is no regional lymph node spread; N1: The cancer has spread to a single lymph node in the true pelvis; N2: The cancer has spread to 2 or more lymph nodes in the true pelvis; N3: The cancer has spread to lymph nodes along the common iliac artery. M categories refer to spread throughout the body—M0 indicates there are no signs of distant spread and M1 that cancer has spread to distant parts of the body, e.g., distant lymph nodes, bones, lungs, liver, etc.

Adapalene may be administered to patients at various stages of bladder cancer. For instance, adapalene may be administered to a patient at Stage 0a (Ta, N0, or M0), Stage 0is (Tis, N0, or M0), Stage I (T1, N0, or M0), Stage II (T2a or T2b, N0, or M0), Stage III (T3a, T3b, or T4a, N0, M0), or Stage IV. In some embodiments, adapalene can be administered to patients exhibiting symptoms of bladder cancer that have a genetic predisposition to bladder cancer. For instance, the patient may be SPARC expression positive or negative, or possess one or more mutations in NFL, p53, MIB-1, FEZ1/LZTS1, PTEN, DBCCR1, CDKN2A/MTS1/P6, ERBB2, CDKN2B/INK4B/P15, TSC1, or HRAS1.

Adapalene may be used for the treatment of bladder cancer in mammals, especially humans, in monotherapy mode or in a combination therapy (e.g., dual combination, triple combination etc.) mode such as, for example, in combination with one or more anti-cancer therapeutics. In some instances, adapalene, either alone or in combination therapy, can be administered to a patient that has already undergone a course of BCG therapy. In some embodiments, the patient may receive BCG treatment 1, 2, 3, 4, 5, 6, 7, 8, 9, 10, 11, or 12 months prior to commencing adapalene treatment. In other embodiments, adapalene, either alone or in combination therapy, can be administered to a patient that has not undergone a course of BCG therapy. In yet further embodiments, adapalene, either alone or in combination therapy, can be administered to a patient that is concurrently undergoing a course of BCG therapy.

Adapalene can be administered to bladder cancer patients also receiving one or more immunotherapeutic agents. Immunotherapies include monoclonal antibodies, i.e., checkpoint inhibitors, and oncolytic virus. Oncolytic viruses are genetically engineered or naturally occurring viruses that selectively replicate in and kill cancer cells without harming the normal tissues. The viruses are modified such that they can replicate in cancerous cells, but not healthy cells.

The term “anti-cancer drug” is used in broad sense to include, but is not limited to, oncolytic viruses, monoclonal antibodies, microtubule inhibitors, topoisomerase inhibitors, platins, alkylating agents, and anti-metabolites. Particular agents include modified adenovirus, modified herpes simplex virus, modified reovirus, modified vaccinia virus, atezolizumab, durvalumab, nivolumab, pembrolizumab, ramucirumab, B-701, MK-6018, ALT-801, paclitaxel, gemcitabine, doxorubicin, vinblastine, etoposide, 5-fluorouracil, carboplatin, oxaliplatin, nedaplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, thiotepa, topotecan, trastuzumab, vincristine, vindesine, and vinorelbine. In certain embodiments, the N-phenyl piperazine can be administered in combination with cisplatin.

In cases of combination therapy, it is possible that a unitary dosage form containing both adapalene and additional anti-cancer agent may be employed. In some instances, the combinations may be provided in form suitable for parenteral application such as but not limited to injection.

In some embodiments, adapalene can be administered as part of a surgical or radiological treatment regime. For instance, a patient may be administered adapalene prior to and/or after undergoing TURBT, partial or radical cystectomy. Likewise, a patient may be administered adapalene prior to and/or after undergoing radiation therapy.

Adapalene can be administered as part of a treatment regime that includes surgical and chemotherapeutic components. The patient, in addition to receiving one or more of the anti-cancer agents identified above, can receive adapalene prior to and/or after undergoing a surgical procedure. In some embodiments, adapalene can be administered as part of a treatment regime that includes radiation therapy and chemotherapeutic components. The patient, in addition to receiving one or more of the anti-cancer agents identified above, can receive adapalene prior to and/or after undergoing radiation therapy. In some embodiments, the chemotherapy includes one or more of cisplatin, fluorouracil, and mitomycin. In other embodiments, adapalene can be administered as part of a treatment regime that includes surgical and immunotherapeutic components. The patient, in addition to receiving one or more of the immunotherapeutic agents identified above, can receive adapalene prior to and/or after undergoing a surgical procedure.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: In Vitro 2D Assay

In the present study the human bladder cancer cell lines BXF 1036, BXF 1218, BXF 1352, 5637 and T24 were used. BXF 1036, BXF 1218 and BXF 1352 were established at Oncotest from the corresponding human patient derived xenograft. T24 was purchased from ATCC (Rockville, Md., USA) and 5637 was from DSMZ (Braunschweig, Germany). Authenticity of cell lines was confirmed at the DSMZ by STR (short tandem repeat) analysis, a PCR based DNA-fingerprinting methodology. Cell lines were routinely passaged once or twice weekly and maintained in culture for up to 20 passages. All cells were grown at 37° C. in a humidified atmosphere with 5% CO₂ in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.1 mg/mL gentamicin (Life Technologies, Karlsruhe, Germany).

The CellTiter-Blue® Cell Viability Assay (#G8081, Promega) was used according to manufacturer's instructions. Briefly, cells were harvested from exponential phase cultures, cells/well depending on the cell line's growth rate. After a 24 h recovery period to allow the cells to resume exponential growth, test compounds were added. Compounds were applied at 9 concentrations in half-log increments in duplicate and treatment continued for 96 h. After 96 h treatment of cells, 20 μL/well CellTiter-Blue® reagent was added.

Following an incubation period of up to four hours, fluorescence (FU) was measured by using the Enspire Multimode Plate Reader (excitation λ=531 nm, emission λ=615 nm). For calculations, the mean values of duplicate/sixfold (untreated control) data were used. Sigmoidal concentration-response curves were fitted to the data points (T/C values) obtained for each cell line using 4 parameter non-linear curve fit (Oncotest Warehouse Software).

The in-vitro anti-tumor activity of adapalene was assessed in five selected human bladder cancer cell lines by using CellTiter-Blue® Adapalene displayed concentration dependent activity with sigmoidal concentration-effect curves in all cell lines tested. Individual IC₅₀ values were in the range from 3.329 μM (T24) to 13.953 μM (BXF 1352), corresponding to 4-fold difference between the most sensitive and most resistant cell line. Overall, the cell lines T24 (IC₅₀=3.329 μM), 5637 (IC₅₀=5.419 μM) and BXF 1218 (IC₅₀=5.998 μM) appeared to be somewhat more sensitive than BXF 1036 (IC₅₀=9.907 μM) and BXF 1352 (IC₅₀=13.953 μM). The reference compound cisplatin showed concentration-dependent activity in all cell lines tested with a geometric mean absolute IC₅₀ value of 8.573 μM. The selectivity profile of cisplatin was quite similar to adapalene, with T24 shown to be the most sensitive and BXF 1352 the most resistant cell line.

TABLE 1

| Cell Line | Adapalene IC₅₀ (μm) | Cisplatin IC₅₀ (μm) |
|---|---|---|
| BXF 1036 | 9.907 | 9.529 |
| BXF 1218 | 5.998 | 7.078 |
| BXF 1352 | 13.953 | 17.059 |
| BXF 5637 | 5.419 | 9.706 |
| BXF T24 | 3.329 | 4.148 |
| Geo. Mean | 6.839 | 8.573 |

### Example 2: In Vitro 3D Assay

The clonogenic assay was carried out in a 96 well plate format using ultra low attachment plates. For each test, cells were prepared as described above and assay plates were prepared as follows: each test well contained a layer of semi-solid medium with tumor cells (50 μL), and a second layer of medium supernatant with or without test compound (100 μL). The cell layer consisted of 2·103 to 3·103 tumor cells per well, which were seeded in 50 μL/well cell culture medium (IMDM, supplemented with 20% (v/v) fetal calf serum, 0.01% (w/v) gentamicin, and 0.4% (w/v) agar. After 24 hours the test compounds were added after serial dilution in cell culture medium, and left on the cells for the duration of the experiment (continuous exposure, 100 μl drug overlay). Every plate included six untreated control wells and drug-treated groups in duplicate at 9 concentrations. Cultures were incubated at 37° C. and 7.5% CO2 in a humidified atmosphere for 8 to 13 days and monitored closely for colony growth using an inverted microscope. Within this period, ex vivo tumor growth led to the formation of colonies with a diameter of >50 μm. At the time of maximum colony formation, counts were performed with an automatic image analysis system (Bioreader 5000-Wa Biosys GmbH). 48 hours prior to evaluation, vital colonies were stained with a sterile aqueous solution of 2-(4-iodophenyl)-3-(4-nitrophenyl)-5-phenyltetrazolium chloride (1 mg/ml, 100 μl/well).

The ability of adapalene and cisplatin to inhibit ex vivo colony formation of cells with the ability to grow anchorage-independently in semi-solid medium was examined in 5 human tumor cell lines of bladder cancer. Adapalene inhibited colony formation in a concentration-dependent manner. The mean relative IC₅₀ value of adapalene was determined as 7.37 μM (mean absolute IC₅₀ value=14.01 μM). Bottom plateaus of the concentration-effect curves of responding tumor models were in the range from 0% to 54%, with a major proportion <10%, indicating clear inhibition of tumor colony growth. Based on relative and absolute IC₅₀ values, above average activity (individual IC₅₀ value<½ mean IC₅₀) was observed against BXF 1218 and BXF T-24, the latter one having a concentration-response relationship with a relative high bottom plateau.

Cisplatin inhibited colony formation in a concentration-dependent manner with a mean relative IC₅₀ value of 9.71 μM (mean absolute IC₅₀ value =9.93 μM). Bottom plateaus of the concentration-effect curves of the responding tumor models were <10%, indicating clear inhibition of tumor colony growth. Based on relative IC₅₀ values, above average activity was observed against 1/8 tumor models (cell line BXF 1036).

TABLE 2

| Cell Line | Adapalene IC₅₀ (μm) | Cisplatin IC₅₀ (μm) |
|---|---|---|
| BXF 1036 | 9.72 | 3.97 |
| BXF 1218 | 4.76 | 5.36 |
| BXF 1352 | 12.80 | 8.65 |
| BXF 5637 | 9.11 | 10.08 |
| BXF T24 | 100.00 | 51.99 |
| Geo. Mean | 14.01 | 9.93 |

### Example 3: Combination Study

The objective of this study was to assess anti-tumor efficacy of adapalene in combination with cisplatin in a 5×5 matrix combination format against various bladder cancer cell lines. Efficacy of the combinations was assessed by measuring anchorage-independent growth and in vitro tumor colony formation using a 3D clonogenic assay in cell lines BXF 1036L, BXF 1218L, and BXF T24. The Bliss independence methodology was used for data analysis, in order to identify possible synergistic effects.

The compounds were tested in bladder cell lines, namely BXF 1036L, BXF 1218L, and BXF T24. Cells lines of BXF 1036L and BXF 1218L were established at Oncotest in Freiburg from the corresponding patient-derived xenografts. BXF T24 cells were obtained from American Type Culture Collection (Rockville, Md., USA). Authenticity of cell lines was confirmed at the DSMZ by STR analysis.

Cell lines were routinely passaged once or twice weekly and maintained in culture for up to 20 passages. Cells were grown at 37° C. in a humidified atmosphere with 5% CO₂ in RPMI 1640 medium (25 mM HEPES, with L-glutamine, Biochrom) supplemented with 10% (v/v) fetal calf serum and 0.1 mg/mL gentamicin. The percentage of viable cells was determined in a Neubauer-hemocytometer using trypan blue exclusion.

The clonogenic assay was carried out in a 96 well plate format using ultra low attachment plates. For each test cells were prepared as described above, and assay plates were prepared as follows: each test well contained a layer of semi-solid medium with tumor cells (50 μl), and a second layer of medium supernatant with or without test compounds (100 μl). The cell layer consisted of 5·10³ to 7.5·10³ tumor cells per well, which were seeded in 50 μl/well cell culture medium (IMDM, supplemented with 20% (v/v) fetal calf serum, 0.01% (w/v) gentamicin, and 0.4% (w/v) agar). After 24 h, the soft-agar layer was covered with 90 μl of the same culture medium without agar, and compounds were added after serial dilution in DMSO and transfer in cell culture medium and left on the cells for the duration of the experiment (continuous exposure, 100 μL total drug overlay). Every plate included six untreated control wells and drug-treated groups. Cultures were incubated at 37° C. and 7.5% CO₂ in a humidified atmosphere for 8 to 13 days and monitored closely for colony growth using an inverted microscope. Within this period, ex vivo tumor growth led to the formation of colonies with a diameter of >50 μm. At the time of maximum colony formation, counts were performed with an automatic image analysis system (CellInsight NXT, Thermo Scientific). 48 hours prior to evaluation, vital colonies were stained with a sterile aqueous solution of 2-(4-iodophenyl)-3-(4-nitrophenyl)-5-phenyltetrazolium chloride (1 mg/ml, 100 μl/well).

The ability of adapalene to inhibit ex vivo colony formation of cells as single agent and in combination with cisplatin was examined in 3 bladder cancer cell lines (BXF 1036L, BXF 1218L and BXF T24). Information about single agent efficacy was derived from monotherapy controls of the 5×5 combination matrix. Adapalene inhibited colony formation of tumor cells with ICso values ranging from 3.44 μM (BXF T24) to 10.8 μM (BXF 1036L). Cisplatin was active against the cell lines BXF 1036L and BXF 1218L (IC₅₀ of 6.09 μM and 6.8 μM, respectively) while being less active against BXF T24 (IC₅₀=77.4 μM). Synergistic effects were recorded for the combination of adapalene with cisplatin in the bladder cancer cell lines BXF 1218L and BXF T24. Additive effects were recorded for the combination of adapalene with cisplatin in the bladder cancer cell line BXF 1036L.

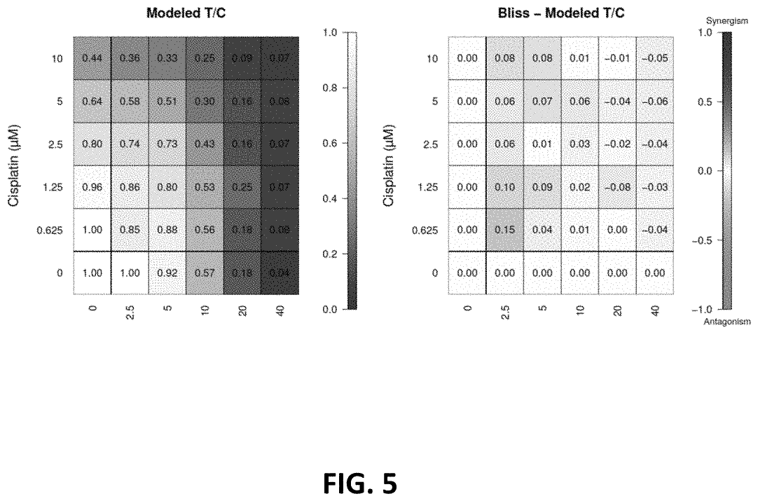

Adapalene and cisplatin inhibited colony formation of BXF 1036L cells seeded in soft agar in a concentration-dependent manner. This is also reflected in the matrix combination, where activity of the different combinations was observed at higher concentrations of both compounds (FIG. 5A).

Bliss independence analysis showed that overall an additive effect of the combinations was obtained, i.e. neither synergy nor antagonism. The color coding of the tiles in the heatmap show, that there is no consistent concentration-dependent effect pointing towards synergy (BI>0.15) or antagonism (BI<−0.15). Thus, the oscillation of individual BI values around 0 rather reflects the variability within the assay (FIG. 5B)

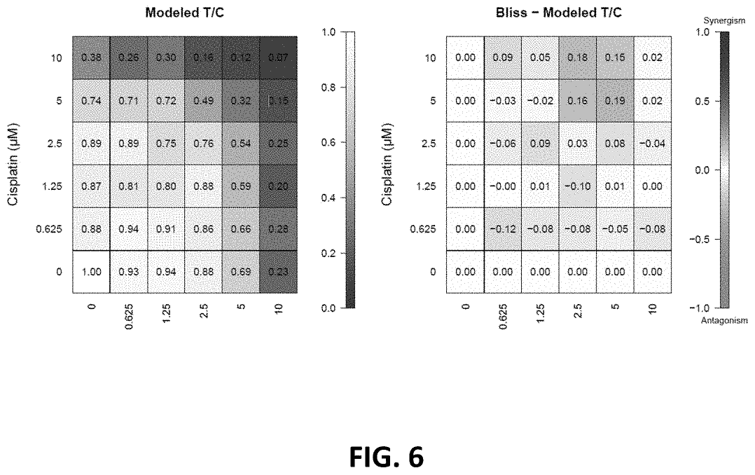

Adapalene and cisplatin inhibited colony formation of BXF 1218L cells seeded in soft agar in a concentration-dependent manner. This is also reflected in the matrix combination, where activity of the different combinations was observed at higher concentrations of both compounds (FIG. 6A).

Bliss independence analysis showed that the combination of adapalene with cisplatin was synergistic at mid concentration levels of adapalene (2.5 μM and 5 μM) and at higher concentration levels of cisplatin (5 μM and 10 μM). The color coding of the tiles in the heatmap show, that there is a consistent effect pointing towards synergy (BI >0.15) (FIG. 6B).

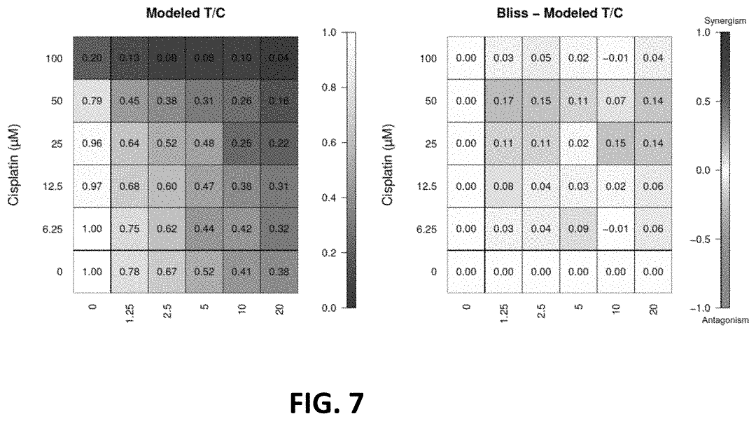

Adapalene and cisplatin inhibited colony formation of BXF T24 cells seeded in soft agar in a concentration-dependent manner. This is also reflected in the matrix combination, where activity of the different combinations was observed at higher concentrations of both compounds (FIG. 7A).

Bliss independence analysis showed that the combination of adapalene with cisplatin was synergistic at several concentrations of adapalene (1.25 μM to 10 μM) and at mid to high concentration levels of cisplatin (25 μM and 50 μM). The color coding of the tiles in the heatmap show, that there is a consistent effect pointing towards synergy (BI>0.15) (FIG. 7B).

### Example 5: Compositions

The manufacturing formula and the process for manufacturing a pharmaceutical composition envisaged under the present invention can be referred herein below:

### Manufacturing Formula 1

| Ingredients | Quantity/Tab (mg) |
|---|---|
| Adapalene | 0.1-10 |
| Microcrystalline cellulose | 10-25 |
| Lactose Monohydrate | 20-80 |
| Croscarmellose Sodium | 5-10 |
| Povidone | 3-10 |
| Aerosil 200 | 1-5 |
| Magnesium Stearate | 1-5 |

1. Adapalene and lactose monohydrate were sifted through specific mesh and loaded into the blender for blending.

2. Croscarmellose sodium, povidone, and colloidal anhydrous silica (Aerosil 200) were separately sifted through specific mesh and collected in suitable container followed by blending.

3. Specific quantity of magnesium stearate was sifted through specific mesh, and sifted magnesium stearate was blended with the blends obtained in the above steps.

4. The lubricated materials obtained above were then finally compressed to form tablets.

### Manufacturing Formula 2

| Ingredients | Quantity Mg/tablet |
|---|---|
| Adapalene | 10-100 |
| Lactose monohydrate (Flow lac 100) | 10-50 |
| Hypromellose (HPMC K4M/K15 M/K100 M) | 20-60 |
| Microcrystallme cellulose | 5-20 |
| Colloidal silicon dioxide (Aerosil 200) | 1-5 |
| Magnesium stearate | 2-8 |
| Purified water | q.s. |

1. Adapalene and lactose monohydrate were sifted through specific mesh, and blended in suitable blender.

2. Hypromellose was dissolved in sufficient quantity of purified water, and stirred continuously for given time.

3. The blended material obtained in step 1 was granulated with the Hypromellose solution prepared above followed by kneading to get the desired consistency of granules.

4. The above obtained granules were then load in suitable equipment for drying.

5. Colloidal silicon dioxide and microcrystalline cellulose were sifted through specific mesh.

6. The materials obtained in step 4 and step 5 above were further mixed and blended for given time followed by lubrication with pre-sifted magnesium stearate.

7. The above obtained lubricated granules were then finally compressed to form tablets.

### Manufacturing Formula 3

| Ingredients | Quantity mg/tablet |
|---|---|
| Adapalene | 100-500 |
| Starch 1500 | 30-150 |
| Microcrystalline cellulose | 50-200 |
| Croscarmellose sodium | 15-45 |
| Magnesium stearate | 5-20 |
| Opadry ready mix | 2.5-10 |
| Purified water | 3-10 |

1. Adapalene, Starch 1500, microcrystalline cellulose and croscarmellose sodium were sifted through specific mesh and were loaded in a suitable blender for blending for given time.

2. Magnesium stearate was separately sifted through specific mesh and was loaded with the above blended materials for lubrication.

3. The lubricated blend was then compressed to form tablets.

4. Film coating solution was prepared using Opadry ready mix with purified water, and the above obtained tablets were then film coated.

### Manufacturing Formula 4

| Ingredients | Quantity mg/tablet |
|---|---|
| Adapalene | 2-200 |
| Starch 1500 | 30-150 |
| Lactose monohydrate (Flow lac 100) | 50-200 |
| Croscarmellose sodium | 5-45 |
| Colloidal silicon dioxide (Aerosil 200) | 1-5 |
| Magnesium stearate | 5-20 |

1. Adapalene, starch 1500, lactose monohydrate (Flow lac 100), colloidal silicon dioxide (Aerosil 200) and croscarmellose sodium were sifted through specific mesh and were loaded in a suitable blender for blending for given time.

2. Magnesium stearate was separately sifted through specific mesh and was loaded with the above blended materials for lubrication.

3. The lubricated blend was then compressed to form tablets.

### Manufacturing Formula 5

| Ingredients | Quantity (% w/w) |
|---|---|
| Adapalene | 10-50 |
| Carbopol 940 | 0.1-100 |
| Disodium edetate | 0.1-5 |
| Methylparaben | 0.1-10 |
| Poloxamer 124 | 1-6 |
| Propylene glycol | 3-12 |
| Sodium hydroxide | 0.1-12 |
| Purified water | q.s. |

1. Adapalane , poloxamer 124 and propylene glycol were mixed and stirred in a suitable container followed by addition of specified quantity of purified water and stirred again.

2. Carbopol 940 was dispersed in the above solution under high-speed stirring.

3. In a separate container disodium edetate, methylparaben, and sodium hydroxide were dissolved in the purified water, and stirred for given time.

4. The solution obtained above in step 2 is added and mixed with the solution in step 3 to form a homogeneous gel.

### Manufacturing Formula 6

| Ingredients | Quantity (% w/w) |
|---|---|
| Adapalene | 0.1-10 |
| Propyl Gallate | 0.02-5 |
| Citric Acid | 0.02-10 |
| Disodium Edetate | 0.02-10 |
| Polysorbate 80 | 0.01-8 |
| Glycerin | 2-50 |
| Methylparaben | 0.1-5 |
| CARBOPOL 981 | 0.5-10 |
| Tromethamine (10% in water) | q.s. to adjust pH |
| Purified water | q.s. |

1. Glycerin and polysorbate 80 were combined and mixed in a suitable container.

2. In a separate container, propyl gallate, citric acid, disodium edetate and methylparaben were dissolved in the purified water.

3. Adapalene was added in the above step 2, and stirred for given time.

4. Carbopol 981 was dispersed in the above step 3, under high-speed stirring.

5. The mixture obtained in step 1 was then added to the above solution obtained in step 4 and stirred continuously until a uniform gel was formed.

6. pH of the gel was adjusted using Tromethamine.

### Manufacturing Formula 7

| Ingredients | Quantity per unit |
|---|---|
| Adapalene | 1-50 mg |
| Ethyl alcohol | 10-20% |
| Benzyl alcohol | 10-20% |
| Polysorbate 80 | 0.1-1.0% |
| α-tocopherol | 0.01-0.1% |
| Castor oil (super refined) | q.s. to 2-5 mL |

1. Benzyl alcohol was added in a suitable tank held at room temperature.

2. Ethyl alcohol was added in the above referenced tank, and the mixture was mixed for a given time.

3. Adapalene was then added to the above referenced tank, and the composition was mixed for a given time until complete dissolution.

4. Polysorbate 80 and a-tocopherol were added in the above composition and were mixed for a given time.

5. Super refined castor oil was then added in the above composition in the tank to approx. 70% of the final volume which was then pressurized with nitrogen followed by mixing for a given time.

6. Super refined castor oil was then further added until the final volume was achieved which was then pressurized with nitrogen and the composition mixed for a given time.

7. The above obtained composition was then filtered through a pre-filter with specific pore size in a suitable vessel followed by filtration in a final filter of specific pore size into a pressure tank fitted with a filling needle under aseptic conditions.

8. The above obtained filtered composition was dispensed in a suitable container, and the filled container was then sealed.

### Manufacturing Formula 8

| Ingredients | Quantity per unit |
|---|---|
| Adapalene | 1-5 mg |
| Polyvinylpyrrolidone (Kollidon 12 PF) | 10-20% |
| Purified Water | q.s. |

1. Adapalene was added in purified water under stirring.

2. Polyvinylpyrrolidone was added to the above solution and stirred for a given time.

3. The above obtained solution was filtered in a suitable container which solution was then lyophilized and filled in ampoule(s).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method for the treatment of bladder cancer in a subject, the method comprising administering to said subject adapalene, or a pharmaceutically acceptable salt thereof, in an amount effective to treat bladder cancer.

2. The method according to claim 1, wherein the patient is diagnosed with Stage 0, Stage I, Stage II, Stage III or Stage IV bladder cancer.

3. The method according to claim 2, wherein adapalene is administered in combination with at least one other cancer therapy.

4. The method according to claim 3, wherein the other cancer therapy comprises surgery, chemotherapy, immunotherapy or radiation therapy.

5. The method according to claim 4, wherein the immunotherapy comprises administering Bacillus Calmette-Guérin vaccine.

6. The method according to claim 5, wherein adapalene is administered subsequent to Bacillus Calmette-Guérin vaccine delivery.

7. The method according to claim 5, wherein adapalene is administered in combination with an immunotherapy comprising a monoclonal antibody or an oncolytic virus.

8. The method according to claim 4, wherein the surgery comprises TURBT, partial or radical cystectomy.

9. The method according to claim 8, wherein adapalene is administered subsequent to surgical treatment for bladder cancer.

10. The method according to claim 8, wherein adapalene is administer prior to the surgical treatment for bladder cancer.

11. The method according to claim 4, wherein the chemotherapy comprises administering an anti-cancer agent comprising one or more microtubule inhibitors, topoisomerase inhibitors, platins, alkylating agents, or anti-metabolites.

12. The method according to claim 11, wherein the anti-cancer agent comprises a platin compound.

13. The method according to claim 12, wherein platin compound comprises cisplatin, carboplatin, oxaliplatin, or nedaplatin.

14. The method according to claim 13, wherein platin compound is cisplatin.

15. A kit comprising adapalene, or a pharmaceutically acceptable salt thereof, and at least one anti-cancer agent comprising one or more monoclonal antibodies, oncolytic viruses, microtubule inhibitors, topoisomerase inhibitors, platins, alkylating agents, or anti-metabolites.

16. A pharmaceutical composition comprising adapalene, or a pharmaceutically acceptable salt thereof, in an amount effective to treat bladder cancer.

17. The pharmaceutical composition according to claim 16, further comprising at least one additional anti-cancer agent.

18. The pharmaceutical composition according to claim 17, wherein the additional anti-cancer agent comprises microtubule inhibitors, topoisomerase inhibitors, platins, alkylating agents, or anti-metabolites.

19. The pharmaceutical composition according to claim 17, wherein the additional anti-cancer agent comprises a platin compound.
